(19) United States
(12) Patent Application Publication
(10) Pub. No.: US 2025/0388596 A1
(43) Pub. Date: Dec. 25, 2025

(54) PROCESS FOR THE EXTRACTION AND PURIFICATION OF TETRODOTOXIN

(71) Applicant: Wex Pharmaceuticals Inc., Vancouver (CA)

(72) Inventors: Meng Zhou, Oakville (CA); Andrea Gambini, Milan (IT); Luca Domenighini, Milan (IT)

(21) Appl. No.: 18/880,674

(22) PCT Filed: Apr. 26, 2023
(86) PCT No.: PCT/CA2023/050562

§ 371 Date: Jan. 2, 2025

Related U.S. Application Data
(60) Provisional application No. 63/358,595, filed on Jul. 6, 2022.

(51) Int. Cl. C07D 491/22; B01D 11/02

(52) U.S. Cl. CPC C07D 491/22; B01D 11/0288

(57) ABSTRACT

A process for extracting TTX from biological material containing TTX, wherein the process comprises steps of extracting with a first solvent, concentrating, washing with a second solvent, concentrating again and purifying by chromatography.

### CROSS REFERENCE TO RELATED APPLICATIONS

The present application claims priority under the Paris Convention to U.S. Application 63/358,595 filed on Jul. 6, 2022, the entire contents of which are incorporated herein by reference.

### FIELD

The present description relates to a process of extraction and purification of tetrodotoxin from biological material.

### BACKGROUND

Tetrodotoxin, also referred to as “TTX”, is a natural toxin occurring in a number of terrestrial and marine macro and microorganisms.

TTX was first isolated in 1909 from the ovaries of globefish. TTX acts as a mediator of voltage gated sodium channels and is a pharmaceutically relevant compound for its potential application as an anaesthetics drug, chemotherapeutic agent and in the treatment of heroine withdrawal effect in rehab regimen (Makarova M. et al., Angewandte Chemie, International Edition 2019, 58(51), 18338-18387). Structures of TTX and of its analogues are reported in the following schemes:

Scheme 1 (TTX and analogues (hemilactal type))

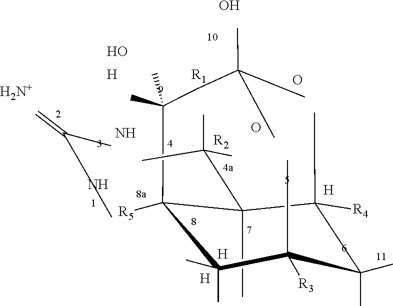

| | R₁ | R₂ | R₃ | R₄ | R₅ |
|---|---|---|---|---|---|
| TTX | H | OH | OH | CH₂OH | OH |
| 4-epiTTX | OH | H | OH | CH₂OH | OH |
| 6-epiTTX | H | OH | CH₂OH | OH | OH |
| 11-deoxyTTX | H | OH | OH | CH₃ | OH |
| 6-epi-11-deoxyTTX | H | OH | CH₃ | OH | OH |
| TTX-a-O- | H | OH | OH | CH₂OH | OOC(CH₂)₂ |
| hemisuccinate | | | | | COO⁻ |
| Chiriquitoxin | H | OH | OH | CH(OH)CH | OH |
| | | | | (NH₃⁺)COO⁻ | |
| 11-norTTX-6(S)-ol | H | OH | OH | H | OH |
| 11-norTTX-6(R)-ol | H | OH | H | OH | OH |
| 11-norTTX-6,6-diol | H | OH | OH | OH | OH |
| 11-oxoTTX | H | OH | OH | CH(OH)₂ | OH |
| TTX-11-carboxyl acid | H | OH | OH | COOH | OH |

Scheme 2 (TTX and analogues (lactone type))

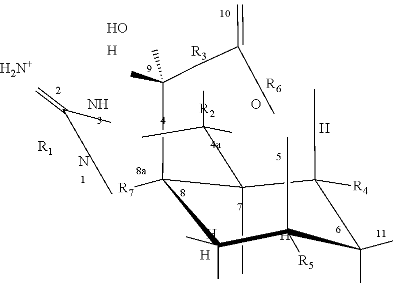

| | R₁ | R₂ | R₃ | R₄ | R₅ | R₆ | R₇ |
|---|---|---|---|---|---|---|---|
| TTX | H | OH | H | CH₂OH | OH | OH | OH |
| 5-deoxyTTX | H | OH | H | CH₂OH | OH | H | OH |
| 5,11-dideoxyTTX | H | OH | H | CH₃ | OH | H | OH |
| 6-epi-5,11- | H | OH | H | CH₃ | OH | H | OH |
| dideoxyTTX | | | | | | | |
| 1-hydroxy-5,11- | OH | OH | H | CH₃ | OH | H | OH |
| dideoxyTTX | | | | | | | |
| 5,6,11-trideoxyTTX | H | OH | H | CH₃ | H | H | OH |
| 4-epi-5,6,11- | H | H | OH | CH₃ | H | H | OH |
| trideoxyTTX | | | | | | | |

Scheme 3 (4,9-anhydro TTX and analogues)

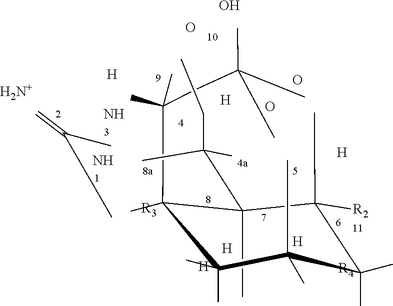

| | R₁ | R₂ | R₃ |
|---|---|---|---|
| 4,9-anhydroTTX | OH | CH₂OH | OH |
| 4,9-anhydro-6- | CH₂OH | OH | OH |
| epi-TTX | | | |
| 4,9-anhydro-6- | H | CH₂OH | OH |
| deoxyTTX | | | |
| 4,9-anhydro-TTX-8-O- | OH | CH₂OH | OOC(CH₂)₂COO⁻ |
| hemisuccinate | | | |
| 4,9-anhydro- | OH | CH₂OCO(CH₂)₂COO⁻ | OH |
| TTX-11-O- | | | |
| hemisuccinate | | | |

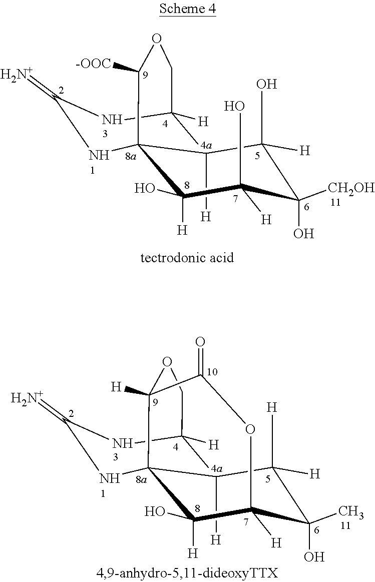

TTX is most commonly obtained through extraction from biological material such as puffer fishes, newts, frogs, toads and microbial fermentation broths (Prathoopa V. et al., Environmental Toxicology and Pharmacology 2016, 36, 1048-1054). Due to the high content of TTX in ovaries, liver and other entrails of several species of puffer fish, these biological materials are typically employed in industrial processes.

In CN112920192, TTX is extracted from the viscera of puffer fish through a process that comprises an ultrasonic extraction with an acidic ethanol solution, decolorization with activated carbon, purification on ion exchange resin column, purification on adsorption resin column and, finally, recrystallization of TTX.

In CN101891751, TTX is extracted from the ovaries of puffer fish through a process that comprises an extraction of the biological material with an acidic alcoholic solution, centrifugation/filtration of the extracting solution, purification on a cationic exchange resin column, concentration of the extraction solution through vacuum distillation and glucose gel column, and isolation of TTX by precipitation with the addition of an organic base. TTX is obtained from this process with a yield of about 0.002% (calculated as g of TTX/g of fish entrails).

In CN102584843, TXX is extracted from the roe or liver of puffer fish through a process that comprises extraction of the biological material with water, denaturation of the proteins in the extraction solution by heat treatment, degreasing with dichloromethane, purification on ion exchange resin column, ultra or nanofiltration to further purify/concentrate the eluate, purification with preparative high performance liquid chromatography and crystallization/precipitation of TTX.

In WO2016061874, TTX is extracted from the liver or roe of globefish through a process that comprises aqueous extraction, separation of the flocculate obtained after the addition of a base to the extraction solution, purification on ion exchange resin column, purification on silica gel column, purification with preparative high performance liquid chromatography after precipitation/crystallization; TTX is obtained with an overall yield about of 0.001% (calculated as g of TTX/g of fish entrails).

In view of the potential pharmaceutical application and, as a consequence, of the market request, there is still the need to develop alternative processes of extraction and purification of TTX.

### BRIEF DESCRIPTION OF THE FIGURES

FIG. 1 is a flow diagram illustrating the steps of the TTX extraction and purification process according to one aspect.

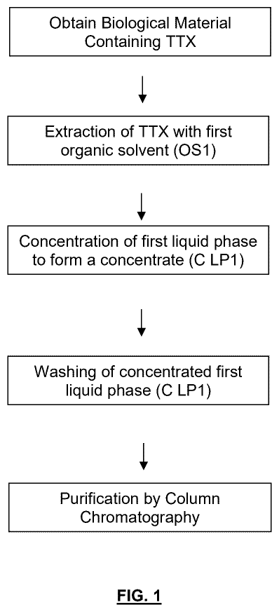

FIG. 2 is a flow diagram illustrating the steps of the TTX extraction and purification process according to one embodiment.

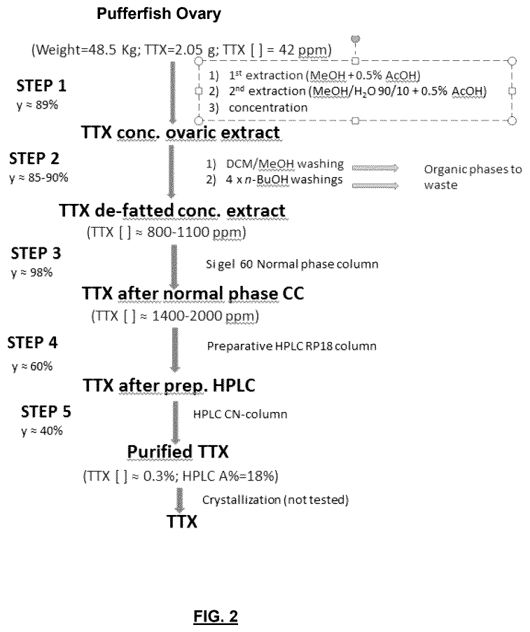

FIG. 3. is a graph illustrating TTX peak elution (crude vs RST (reference substance)) through the RP18 HPLC method with sodium 1-heptanesulfonate in the mobile phase (analytical scale [symbol] =4.6 mm).

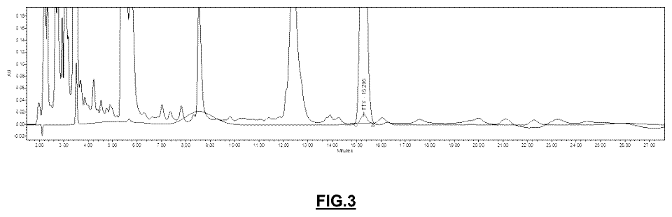

FIG. 4. are graphs illustrating TTX purities (HPLC A %) comparison between the intermediates after the prep. RP18-HPLC step (a) and after the CN-HPLC purification (b). The corresponding data is shown in the adjacent table.

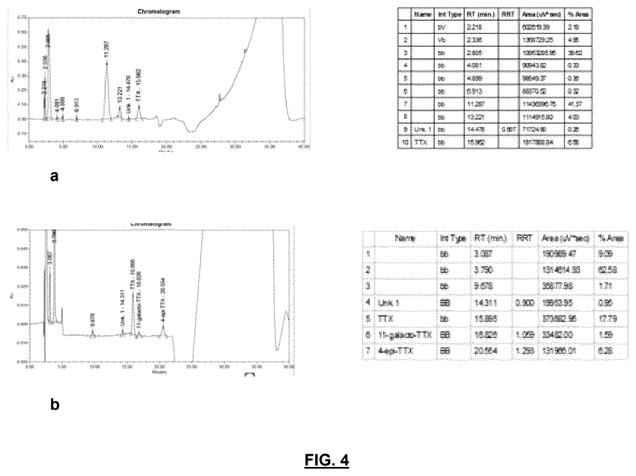

FIGS. 5a-c are photographs of the ovary paste in the reactor undergoing stirring.

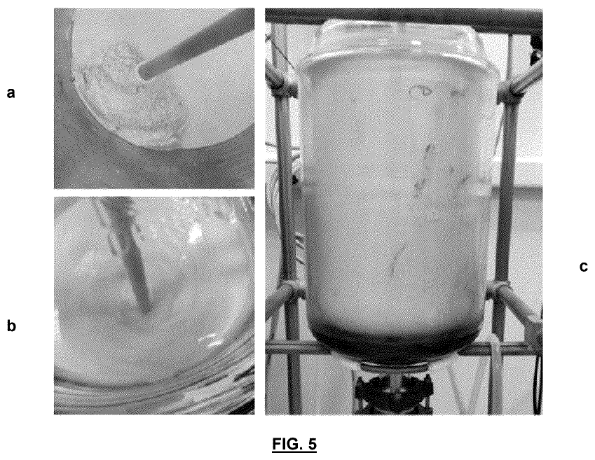

### DESCRIPTION

As described herein there is provided a process for the extraction and purification of TTX from biological material.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein the term “about” is synonymous with “approximately” and is used to provide flexibility to a numerical value, or to the start-and endpoints of a range, by providing that a given value may be “a little above” or “a little below” the value stated. “About” can mean, for example, within three or more than three standard deviations. “About” can mean within a percentage range of a given value. For example, the range can be ±1%, ±5%, ±10%, ±20%, ±30%, ±40% or ±50% of a given value. “About” can mean with an order of magnitude of a given value, for example, within 2-fold, 3-fold, 4-fold, or 5-fold of a value. However, it is to be understood that even when a numerical value is characterized herein by the term “about”, express support shall be provided at least for the exact numerical value as though the term “about” were not present. In one aspect, the term about will be understood to encompass a range ±10% of the respective value.

The term “adsorption chromatography” refers to the analytical separation of a chemical mixture based on the interaction of the adsorbate with the adsorbent. The mixture of gas or liquid is separated when it passes over the adsorbent bed that adsorbs different compounds at different rates. Examples of suitable adsorbents are silica gel, modified silica gel. cellulose microcrystalline, and alumina.

The term “and/or” can mean “and” or “or”.

The term “biological material” as used herein refers to any kind of micro or macro organism containing TTX. Biological material may include the whole organism or a portion of the organism such as tissues obtained from such organisms. The tissues may include ovary, liver, gonad, muscle, skin, or other tissue(s) of the organism that contains TTX.

The terms “comprise”, “comprises”, “comprised”, or “comprising” may be used in the present description. As used herein (including the specification and/or the claims), these terms are to be interpreted as specifying the presence of the stated features, integers, steps, or components, but not as precluding the presence of one or more other feature, integer, step, component, or a group thereof as would be apparent to persons having ordinary skill in the relevant art. Thus, the term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

The phrase “consisting essentially of” or “consists essentially of” will be understood as generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open-ended term, such as “comprising” or “including”, it will be understood that direct support should be afforded also to “consisting essentially of” language as well as “consisting of” language as if stated explicitly and vice versa. In essence, use of one of these terms in the specification provides support for all of the others.

The term “dry residue” refers to the inorganic and/or organic solid material obtained after evaporation of a liquid phase or a washing phase. The amount of a dry residue can be measured by weighing on a scale or with a thermogravimetric apparatus.

The term “extraction solid residue” refers to the residual biological material obtained after extraction with an organic solvent.

The term “liquid phase” defines any liquid, for example a solution, a suspension, an emulsion, a slurry, a foam or the like obtained in one or more steps of the process that is collected and further processed.

The term “organic solvent” as used herein refers to a carbon based solvent capable to dissolving or dispersing one or more other substances. In one aspect, the organic solvent is selected from acetone, acetonitrile, benzene, n-butanol, n-butyl acetate, carbon tetrachloride, chloroform, cyclohexane, 1,2-dichloroethane dichloromethane, diethyl ether, isopropyl ether, dimethylformamide, dimethyl sulfoxide, dioxane, ethanol, ethyl acetate, heptane, hexane, isooctane, isopropanol, methanol, methyl ethyl ketone, methyl tert-butyl ether, nitromethane pentane, 1-propanol, tetrahydrofuran, toluene, trichloroethylene, xylene or a mixture thereof. The term “organic solvent” as used herein may also refer to a mixture of a water miscible organic solvent mixed with water.

The term “washing phase” defines any liquid, for example a solution, a suspension, an emulsion, a slurry, a foam or the like obtained in one or more steps of the process that is collected and discharged.

The term “recovery” or “percent recovery” refers to the amount of TTX obtained from an extraction or purification process relative to the total amount TTX available in the starting material. The percent recovery may be determined for the entire process where the amount of TTX in the final product is defined relative to the amount of TTX in the first starting material or it may be determined for an individual step of the process where the amount of TTX is defined relative to the amount of TTX available in the material obtained after the preceding step of the process.

The term “yield” or “percent yield” refers to the amount of TTX obtained from an extraction process relative to the total biomass of the starting material. As the percentage yield of TTX is measured relative to total biomass of starting material this measurement is affected by both the concentration of TTX available in the biomass and the effectiveness of the extraction and purification process.

The Applicant has developed a process for the extraction and purification of TTX from biological material, wherein the biological material is extracted with an organic solvent. In one embodiment the organic solvent contains water in an amount not higher than 40%.

In one aspect, the process comprises the following steps:
a) extraction of a biological material with a first organic solvent (OS 1) to provide a first extraction solid residue (“ESR 1”) and a first liquid phase (LP 1);
b) concentration of LP 1 to provide a concentrated liquid phase (C-LP 1)
c) washing of C-LP 1 with a second organic solvent (OS 2) to provide a second liquid phase (LP 2) and a washing phase (WP 1);
d) concentration of LP 2 to provide a pre-purified TTX (PP-TTX)
e) purification of PP-TTX by silica gel column chromatography to provide crude TTX (C-TTX).

In the process described above, step (a) may be repeated (step (a1)).

In an embodiment of the process described above OS 1 and OS 2 may be different from each other.

In another aspect, the process comprises the following steps:
a) extraction of a biological material with a first organic solvent (OS 1) to provide a first extraction solid residue (ESR 1) and a first liquid phase (LP 1);
a1) extraction of the extraction solid residue (ESR 1) with a further organic solvent (OS 3) to provide a further extraction solid residue (ESR 2) and a further liquid phase (LP 3);
a2) pooling LP1 and LP 3 to provide a combined liquid phase (LP 4);
b) concentration of LP 4 to provide a concentrated liquid phase (C-LP 4);
c) washing of CLP 4 with a second organic solvent (OS 2) to provide a second liquid phase (LP 2) and a washing phase (WP 1),;
d) concentration of LP 2 to provide a pre-purified TTX (PP-TTX);
e) purification of PP-TTX by silica gel column chromatography to provide crude TTX (C-TTX); and, optionally
f) purification of C-TTX by preparative high performance liquid chromatography to provide purified TTX (P-TTX).

In an embodiment of the process there is a further step g) comprising a further preparative HPLC purification step to reduce the content of the residual ion pairing agent in the P-TTX.

In an embodiment of the process described above, OS 3 is different from OS 1.

In a further embodiment OS 2 is different from one or both of OS 3 and OS 1.

Advantages of the extraction and purification process are discussed in the following sections.

The process can be carried out using any tissue from the organism from which TTX is to be extracted. This included the whole body, the skin, the flesh, the entrails or the eggs of any kind of biological material that contains TTX. Examples of organisms from which TTX can be extracted include but are not limited to, puffer fishes (Takifugu spp., Tetradon spp., Lagocephalus spp., Spheroides spp., Arothon spp.), gastropods (Rapana spp., Nassarius spp., Oliva spp.) flatworms or hammerhead worms (Bipalium spp., Notoplana spp., Stylochus spp., Planocera spp.), octopus (Hapalochleana spp.), toads (Atelopus spp.), newts (Taricha spp., Notophthalmus spp, Cynops spp., Pachytriton spp., Paramesotriton spp., Laotriton spp.), crabs (Tachypleus spp., Atergatis spp.), mussels (Mytilus spp.), starfishes (Astropecten spp.) and fermentation broths of proteobacteria (Vibrio spp., Pseudomonas spp., Serratia spp., Roseobacter spp., Aeromonas spp., Shewanella spp., Marinomonas spp., Plesiomonas spp., Alteromonas spp., Actinobacter spp., Caulobacter spp.), actinobacteria (Microbacterium spp., Micrococcus spp., Kytococcus spp., Cellulomonas spp., Actinomycete spp., Nocardiopsis spp., Streptomyces spp.), firmicutes (Bacillus spp., Lysinibacillus spp.) and bacteriodetes (Flavobacterium spp., Tenacibaculm spp).

In one aspect each of the organic solvents, OS 1 and OS 3, used in steps a) and a1) of the process, is selected from: acetone, acetonitrile, dimethylformamide, dimethyl sulfoxide, dioxane, ethanol, isopropanol, methanol, 1-propanol and tetrahydrofuran and mixtures thereof with water. In a preferred aspect the organic solvent is selected from acetone, methanol and ethanol and mixtures thereof with water. In a further preferred aspect, the organic solvent is methanol or a mixture of methanol and water.

In an embodiment, an organic acid is added to OS 1 and/or OS 3, wherein the organic acid is: formic acid, acetic acid, trifluoroacetic acid, propionic acid, pentanoic acid, hexanoic acid, butyric acid, sorbic acid, lactic acid, citric acid, ascorbic acid, fumaric acid, malic acid, tartaric acid, oxalic acid, citric acid, gluconic acid, glutaric acid, glutamic acid, benzoic acid, benzylic acid, acetylsalicylic acid, cinnamic acid, and/or gallic acid; preferably, acetic acid. The amount of organic acid added is about 0.01 to about 5% of the total volume of OS 1 and/or OS 3; preferably, about 0.1 to about 2.5%; more preferably, about 0.5 to about 1%.

The amount of OS 1 and/or OS 3 may independently be 1-10 V, where V is Litres of solvent per kilogram of biological material, or 3-6 V. In a preferred aspect, the amount of solvent is 5 V.

Each of steps a) and a1) can, independently, be repeated once, or any number of times. In one aspect, such steps are performed twice, up to 5 times, or up to 10 times. As will be understood, such multiple washings would serve to increase the extraction of the desired product.

In another aspect the water content of OS 1 and/or OS 3 is not more than 40%, than 35%, than 30%, than 25%, than 20%, than 15%, than 10%, than 5% of their volume; more preferably, not more than 20%, than 15%, than 10%, than 5%; even more preferably; not more than 10%, than 5%.

The use of an organic solvent with a content of water not exceeding 40% of its total volume is particularly advantageous. Indeed, the use of water, disclosed in CN102584843 and WO2016061874, or the use of an organic solvent with a content of water greater than 40% of the total volume, disclosed in CN101891751, for the extraction of biological material involves several drawbacks. For example, the separation of the extraction solid residue from an aqueous liquid phase is challenging either by filtration, due to the extremely low speed of filtration and the possible formation of clogging, or by decantation. Moreover, the more water that is used for the extraction of the biological material, the greater is the amount of extracted proteins. When greater amounts of protein are extracted, a denaturation step is required and, as in CN102584843, it usually involves a heat treatment. However, TTX, which is not stable at high temperature, can be degraded by heat treatment. Furthermore, as TTX occurs in the biological material in extremely low quantities, in order to efficiently extract it, high volumes of solvent are needed. Moreover, the use of organic solvents in which the water content is not more than 40% is advantageous, because the removal of organic solvents, for example, through vacuum distillation, is easier than the removal of water, and is less costly in time and energy.

Another unexpected finding is related to the stability of TTX in organic solvent comprising less than 40% water. As shown in Table 1, when, for example, LP 1 is stored up to 15 days at room temperature (r.t.) or low temperature (I.t., 2-8° C.) epimerization of 4-epi-TTX, into TTX occurs. Without being bound to theory, the epimerization is believed to be promoted by the presence of methanol and acetic acid in the solution.

TABLE 1

| | TTX | | TTX | | TTX | |
|---|---|---|---|---|---|---|
| | concn. | A % TTX/ | concn. | A % TTX/ | concn. | A % TTX/ |
| Sample | (μg/mL) | A % e-TTX | (μg/mL) | A % e-TTX | (μg/mL) | A % e-TTX |
| | t = 0 | | t = 5 days | | t = 15 days | |
| Example 4 | 3.3 | — | 2.9 | 86.3/10.0 | 3.2 | 94.4/3.5 |
| (r.t.) | | | | | | |
| Example 4 | 3.3 | — | 2.9 | 86.6/9.9 | 3.3 | 92.5/4.7 |
| (l.t.) | | | | | | |
| | t = 0 | | t = 6 days | | t = 15 days | |
| Example 6 | 5.2 | — | 4.0 | 88.6/9.7 | 4.0 | 95.9/3.1 |
| (r.t.) | | | | | | |
| Example 6 | 5.2 | — | 4.0 | 86.1/9.4 | 4.3 | 92.8/3.6 |
| (l.t.) | | | | | | |

In step b), LP 1 or LP 4 may be concentrated through distillation, vacuum distillation, ultra or nanofiltration; preferably, vacuum distillation. After step b), the volume of LP 1 or LP 4 is reduced by 80%, 85%, 90%, 95%, 98% or 99% of their starting volume; preferably, between 90-95%.

By means of step c), the amount of undesired compounds such as, but not limited to, organic and inorganic salts, fatty acids, waxes, polysaccharides, amino acids contained, in C-LP 1 or C-LP 4 is reduced. In the prior art, the first purification step is usually a chromatographic purification involving the use of ion exchange or adsorption resins. Even if through such methods a partially purified solution is achieved, the use of resins has several drawbacks; for example, a large amount of solvent is required to wash and activate the resins, then to elute the desired compounds and finally to regenerate the resins, with a consequently large amount of solvents to concentrated or discharge. Contrary to the prior art, the process described herein does not contain any step of chromatographic purification involving the use of a resin.

Moreover, the Applicant has observed that in the attempt of purifying, for example, LP 4 with an ion exchange or an adsorption resin, as reported in Table 2, TTX was co-eluted after 1 bed volume with most of its impurities.

TABLE 2

| Stationary phase | Eluent | Load | Result |
|---|---|---|---|
| Cation (H+) exchange | H₂O | LP 4, | TTX co-eluted |
| resin | (pH = 6) | pH = 8.5 | with impurities |
| (CM Sepharose Fast Flow) | | | |
| (25 parts vs dry residue from | | | |
| LP4) | | | |
| Cation (H+) exchange | H₂O | LP 4, | TTX co-eluted |
| resin | (pH = 6) | pH = 4.1 | with impurities |
| (CM Sepharose Fast Flow) | | | |
| (25 parts vs dry residue from | | | |
| LP4) | | | |
| Adsorption resin | H₂O | LP 4, | TTX co-eluted |
| (XAD 7 HP) | (pH = 8.8) | pH = 8.5 | with impurities |
| (20 parts vs dry residue from | | | |
| LP4) | | | |
| Adsorption resin | H₂O | LP 4, | TTX co-eluted |
| (XAD 7 HP) | (pH = 5.5) | pH = 4.1 | with impurities |
| (40 parts vs dry residue from | | | |
| LP4) | | | |
| Adsorption resin | H₂O | LP 4, | TTX co-eluted |
| (HP20) | (pH = 1.78) | PH = 2.8 | with impurities |
| (25 parts vs dry residue from | | | |
| LP4) | | | |
| Adsorption resin | H₂O | LP 4, | TTX co-eluted |
| (HP20) | (pH = 8.6) | pH = 8.5 | with impurities |
| (50 parts vs dry residue from | | | |
| LP4) | | | |

In one aspect, in step c) OS 2, is selected from: benzene, n-butanol, n-butyl acetate, carbon tetrachloride, chloroform, cyclohexane, 1,2-dichloroethane dichloromethane, diethyl ether, diisopropyl ether, ethyl acetate, heptane, hexane, isooctane, methanol, methyl ethyl ketone, methyl tert-butyl ether, nitromethane pentane, toluene, trichloroethylene, xylene; preferably, methanol, dichloromethane, hexane and butanol and mixtures thereof. Optionally, in step c) C-LP 1 or C-LP 4 is subjected to more washings in which OS 2 is independently the same or a different organic solvent or mixture of solvents for each individual washing. In one embodiment step c) comprises up to 12, 11, 10, 9, 8, 7, 6, 5, 4, 3 or 2 washings. As will be understood, such multiple washings may be used to increase the extraction of TTX.

In step c) the amount of OS 2 used for each washing is comprised of between 0.1-2 volumes per volume of C-LP 1 or C-LP 4 or the liquid phase obtained by the previous washing. In another aspect, a ratio of 0.5-1 volume may be used.

In an embodiment, washings are carried out with hexane. It was observed that this reduces the dry residue up to 5%, without loss of TTX.

Unexpectedly, when LP 2 is stored up to 15 days at room temperature (r.t.) or low temperature (I.t., 2-8° C.) not only epimerization of e-TTX into TTX occurs, but also an increase in the concentration of TTX is observed, as reported in Table 3:

TABLE 3

| | TTX | | TTX | | TTX | |
|---|---|---|---|---|---|---|
| | concn. | A % TTX/ | concn. | A % TTX/ | concn. | A % TTX/ |
| Sample | (μg/mL) | A % e-TTX | (μg/mL) | A % e-TTX | (μg/mL) | A % e-TTX |
| | t = 0 | | t = 9 days | | t = 15 days | |
| Example 10 | 23.2 | 86.5/9.7 | 29 | 93.7/3.9 | 37.6 | — |
| (r.t.) | | | | | | |
| Example 10 | 23.2 | 86.5/9.7 | 27 | 90.3/5.3 | 39 | 92.0/3.5 |
| (l.t.) | | | | | | |
| | t = 0 | | t = 8 days | | t = 14 days | |
| Example 11 | 30.8 | 88.1/10 | 31.7 | 92.9/3.9 | 46.7 | 92.8/3.6 |
| (r.t.) | | | | | | |
| Example 11 | 30.8 | 88.1/10 | 28.9 | 95.5/3.9 | 46.7 | 91.4/3.4 |
| (l.t.) | | | | | | |

Usually C-LP 1 or C-LP 4 shows a floating solid substance on the surface, probably due to flocculation of part of the undesired compounds. The washings for example, with a mixture dichloromethane and methanol allows for removal of the floating solid with a loss of TTX of less than 3%, preferably less than 2%. In one embodiment the ratio of dichloromethane to methanol is in the range of 99:1-70:30; preferably 98:2-85:15; more preferably 95:5-90-10. Repeated washings with another organic solvent selected from dichloromethane, ethyl acetate, hexane, diethyl ether, n-butanol, more preferably n-butanol, allow for further reduction of the dry residue content up to 40%, preferably up to 25%. The extent of reduction of dry residue content depends on the number washings, the pH and the concentration of C-LP 1 and/or C-LP 4. In one embodiment, in order to reduce the loss of TTX, the pH of C-LP 1 and/or C-LP 4 is in the range of pH=3-6; preferably, pH=3.5-5.5; more preferably; pH=4-4.5.

TABLE 4

| | TTX loss | dry residue loss |
|---|---|---|
| Diluted solution (pH = 8.5) | −30% | −10% |
| Diluted solution (pH = 4.1) | −15% | −35% |
| Concentrated solution | −3% | −22% |
| (pH = 4.3) | | |

Preferably, to reduce the loss of TTX (i.e., to improve extraction efficiency), the solvent washing in step (c) may be repeated at least once, and preferably at least four times. An example of this is illustrated in Table 5, which provided data using n-butanol as the solvent, and where the washing is performed twice and four times.

TABLE 5

| Number of n-butanol | | |
|---|---|---|
| extraction | TTX loss | dry residue loss |
| 2 | −2.5% | −16.5% |
| 4 | −0.5% | −22% |

LP 2 obtained from step c) is concentrated in step d). In step d), LP2 may be concentrated by the same methods as described for step b), including distillation, vacuum distillation, ultra or nanofiltration; preferably, vacuum distillation. Preferably, LP2 is concentrated to dryness through vacuum distillation or freeze drying, to provide PP-TTX.

Advantageously, the PP-TTX obtained in step d), contrary to LP 1 or LP 4, can be purified by chromatography over, for example, silica gel, as discussed in the following section. Other column media, such as, reverse phase RP18, cationic and anionic resins, molecular exclusion resin and charcoal, were also tried but did not provide satisfactory results.

In step e) silica gel for chromatographic purification may have a granulometry in the range of 10-500 μm; preferably, 60-200 μm. Moreover, the ratio between silica gel and PP-TTX is comprised between 10-200; preferably 50-150; more preferably 120.

In step e) one or more solvents selected from methanol, ethanol, propanol, iso-propanol, acetone, acetonitrile, water, and ethyl acetate may be used in the mobile phase. Preferably, the solvent comprises a combination of methanol, water and ethyl acetate. In a further preferred aspect of the description, the ratio of ethyl acetate to methanol to water is 10:10:3, 5:5:1, or 4:4:1.

In another aspect, an organic acid, such as acetic acid, formic acid, and/or trifluoroacetic acetic acid, is added to the mobile phase. Preferably, the organic acid is acetic acid. The quantity of acid added to the mobile phase may be between 0.1-10% of the volume of the mobile phase; and preferably 1-5%.

Table 6 provides examples of solvent mixtures that were used for chromatographic separation in step e).

TABLE 6

| Stationary phase | Eluent | Load | Result |
|---|---|---|---|
| Si-gel 60 | MeOH/H₂O 1/1 | C-LP 4, as a | TTX co-eluted with |
| (60 parts vs dry | (+NH₃ to pH = 8.7) | MeOH/H₂O 1/1 mixture | impurities in the |
| residue from C-LP4) | | basified to pH = 8.4 | head fractions |
| Si-gel 60 | EtOAc/MeOH/H₂O | C-LP 4, as | 30% of TTX eluted |
| (90 parts vs dry | 10/10/3 (+AcOH | EtoAc/MeOH/H₂O | in the head fractions; |
| residue from C-LP4.) | added after 5BV) | 10/10/3 slurry | 70% of TTX eluted |
| | | | after AcOH adding |
| Si-gel 60 | EtOAc/MeOH/1/1 | C-LP 4, basified | TTX eluted in the |
| (60 parts vs dry | | to pH = 8.5 | head fractions, |
| residue from C-LP4) | | | but 50% of the |
| | | | impurities retained |
| | | | by the column |
| Si-gel 60 | EtOAc/MeOH/1/1 + | C-LP 4, as | 50% of TTX eluted |
| (60 parts vs dry | EtOAc/MeOH/H₂O | MeOH slurry. | in the head fractions, |
| residue from C-LP4) | 5/5/1 + 1% of AcOH | | 50% eluted after |
| | (added after 5BV) | | AcOH addition |
| Si-gel 60 | EtOAc/MeOH/1/1 + | Pre-purified TTX, as | TTX eluted only |
| (120 parts vs dry | EtOAc/MeOH/H₂O | MeOH slurry. | after AcOH addition. |
| residue of pre- | 4/4/1 + EtOAc/MeOH/H₂O | | |
| purified TTX) | 4/4/1 + 1% of AcOH | | |
| | (added after 12BV) | | |

Step e) provides C-TTX. In optional step f), the process further comprises a preparative HPLC step for the purification of C-TTX. For this step, one or more solvents selected from methanol, ethanol, propanol, iso-propanol, acetone, acetonitrile, water, and ethyl acetate are used as mobile phase. In a preferred embodiment, the mobile phase further contains an organic acid selected from formic acid, acetic acid, and trifluoroacetic acid.

In one aspect, the solvent used in step f) may further comprise an ion pairing agent selected from sodium 1-propanesulfonate, sodium 1-butanesulfonate, sodium 1-pentansulfonate, sodium 1-hexanesulfonate, sodium 1-heptanesulfonate, sodium 1-octanesulfonate, sodium 1-nonanesulfonate, sodium 1-decanesulfonate, sodium 1-undecanesulfonate, sodium 1-dodecanesulfonate, sodium 1-tridecanesulfonate, sodium dodecyl sulfate, tetraethylammonium hydroxide, tetrapropylammonium hydroxide, tetrabutylammonium hydroxide, tetrabutylammonium bromide, tetrabutylammonium chloride, tetrabutylammonium phosphate, tetrabutylammonium hydrogen sulfate, dodecyltrimethylammonium chloride, and tetra (decyl) ammonium bromide.

In one aspect, the organic acid is trifluoroacetic acid and the ion pairing agent is sodium 1-heptanesulfonate.

In step f), the concentration of the organic acid may be between 1-100 ppm; preferably between 15-85 ppm; more preferably, between 40 and 60 ppm. Further, in step f), the concentration of ion pairing agent is preferably in the range of 0.5-12 mM; more preferably, 4-10 mM; still more preferably; 8 mM.

Step f) allows for further extraction of TTX following step e). The percent recovery of TTX achieved in step f), from the C-TTX of step e), is more than 5%, than 10%, than 15%, than 20%, than 25%, than 30%, than 35%, than 40%, than 45%, than 50%, than 55%, than 60%, than 65%, than 70%, than 75%, than 80%, than 85%, than 90%, than 95% or than 99%.

Optionally, the above-noted process includes a step g), comprising a further preparative HPLC purification step to reduce the content of the residual ion pairing agent in the P-TTX 1. For this purpose, one or more solvents selected from methanol, ethanol, propanol, iso-propanol, acetone, acetonitrile, water and ethyl acetate may be used as the mobile phase. Preferably, the mobile phase further contains an organic acid selected from formic acid, acetic acid and trifluoroacetic acid and an organic base selected from triethylamine, diethylamine and ammonia. Preferably the organic acid is formic acid and the organic base is ammonia.

In one aspect, the concentration of organic acid is between 50-200 ppm; preferably between 80-180 ppm; more preferably, between 140 and 160 ppm; the concentration of organic base is 100-1500 ppm; preferably, 300-1000 ppm; more preferably, 600 ppm.

Table 7 illustrates this examples of this further step g).

TABLE 7

| Stationary phase | Mobile phase | Outcome |
|---|---|---|
| Zorbax ™ 300SB- | Isocratic = Water + 50 ppm TFA | TTX and Na-1-heptanesulfonate |
| C18, 250 × 4.6 mm | | eluted in the head fraction |
| Zorbax ™ 300SB-C | Isocratic = Water + NH3/HCO2H | TTX and Na-1-heptanesulfonate |
| 18, 250 × 4.6 mm | (pH = 4.35) | eluted in the head fraction |
| Zorbax ™ 300SB- | Isocratic = Water + NH3/AcOH | TTX and Na-1-heptanesulfonate |
| C18, 250 × 4.6 mm | (pH = 6.00) | eluted in the head fraction |
| Discovery RP-Amide- | Gradient = Water/MeCN + | TTX and Na-1-heptanesulfonate |
| C16 column | NH3/HCO2H buffer | eluted in the head fraction |
| 250 × 4.6 mm | | |
| CN column | Gradient = Water/MeCN + | TTX and Na-1-heptanesulfonate |
| 250 × 4.6 mm | NH3/HCO2H buffer | eluted in the head fraction |
| | (Normal phase gradient) | |
| CN column | Gradient = Water/MeCN + | TTX peak eluted after the head |
| 250 × 4.6 mm | NH3/HCO2H buffer | fraction |
| | (Reverse phase gradient) | |

With step g), the content of the ion pairing agent may be reduced by 50%, 55%, 60%, 65%, 70%, 75%, 80%, 85%, 90%, 95%, 99% with respect to the initial amount after a number of HPLC runs, preferably not less than 3, 5, 8, 12, 17, 23, 25 or 30 runs.

### EXAMPLES

### Example 1—Extraction with MeOH+AcOH 1% v/v

0.20 kg (TTX=10 ppm) of pufferfish ovaries were defrosted and cut in small pieces after approx. 45 minutes, then ground by meat dicer affording 0.14 kg of ovary paste. The paste was then transferred in a 1 L reactor and 5 volumes (V) (where V is 1 L/Kg of ovary paste) of solvent (MeOH+AcOH 1% v/v) was added for extraction. The mixture was stirred for 30 minutes. After that, it was left in the reactor and the complete separation of the phases was observed within 10 minutes. Despite this separation, the solution was discharged from the reactor then filtered on a 240 mm Buchner equipped with a por. 42 paper filter. A clear solution was collected after the filtration. The solid residue (approx. 100 g) was subjected to a second extraction step (i.e., Step a1). For the second extraction, 5 V of solvent (MeOH+AcOH 1% v/v) was added and the mixture was stirred for 30 minutes. After the mixing, the separation of the phases was observed within 30 minutes, then the mixture was discharged and filtered using a smaller Buchner (110 mm) equipped with a por. 42 filter paper. No significant issues were observed during the filtration step, and 90 g of wet residue were collected after the filtration. After two extractions, the concentration of TTX in the combined extracting solutions was approx. 9.3 ppm and in the wet residue is not detected. TTX recovery R %=93% (calculated as [ppm of TTX in the extracting solution] /[ppm of TTX in the ovaries]).

### Example 2—Extraction with EtOH+AcOH 1% v/v

0.61 kg (TTX 1=84 ppm) of pufferfish ovaries were defrosted and cut in small pieces after approx. 45 minutes, then ground by meat dicer affording 0.61 kg of ovary paste. The paste was then transferred in a 1 L reactor and 5 volumes (V) of solvent (EtOH+AcOH 1% v/v) was added for extraction. The mixture was stirred for 60 minutes at 200 rpm, after that it was discharged. After 90 minutes, the separation of the phases was observed. Despite this separation, the solid was again suspended by agitation of the mixture and the mixture was filtered on a 240 mm Buchner equipped with a por. 42 paper filter. A clear solution was collected after the filtration. The solid residue (approx. 370 g) was subjected to the second extraction step (i.e., step a1). For the second extraction, 5 volumes (V) of solvent (EtOH+AcOH 1% v/v) was added and the mixture was stirred for 60 minutes at 200 rpm. After the mixing, the separation of the phases was observed within 60 minutes, then the mixture was filtered on a 240 mm Buchner equipped with a por. 42 paper filter. A clear solution was collected after the filtration. No significant issues were observed during the filtration step, and 315 g of wet residue were isolated. After two extractions, the concentration of TTX in the combined extracting solutions was approx. 36.4 ppm and in the wet residue was 27 ppm. TTX recovery R %=93% (calculated as [ppm of TTX in the extracting solution]/[ppm of TTX in the ovaries).

### Example 3-Extraction with Acetone +AcOH 1% v/v

0.61 kg of ovary (TTX=63 ppm) were defrosted and cut in small pieces after approx. 45 minutes, then ground by meat dicer affording 0.61 kg of ovary paste. The paste was then transferred in a 1 L reactor and 5 volumes (V) of solvent (acetone+AcOH 1% v/v) was added for extraction. The mixture was stirred for 60 minutes at 200 rpm, after that it was discharged. After 15 minutes, the separation of the phases was observed. Despite this separation, the solid was again suspended by agitation of the mixture and the mixture was filtered on a 240 mm Buchner equipped with a por. 42 paper filter. A clear solution was collected after the filtration. The solid residue (approx. 353 g) was subjected to the second extraction step (“step a1”). For the second extraction step, 5 volumes (V) of solvent (acetone+AcOH 1% v/v) was added and the mixture was stirred for 60 minutes at 200 rpm. After the mixing, the separation of the phases was observed within 120 minutes, then the mixture was filtered on a 240 mm Buchner equipped with a por. 42 paper filter. A clear solution was collected after the filtration. No significant issues were observed during the filtration step, and 315 g of wet residue were isolated. After the two extractions, the recovery of TTX is R %=5.2% (TTX=3.27 ppm).

### Example 4-Extraction with MeOH+AcOH 0.5% v/v

0.51 kg of ovary (TTX=48 ppm) were defrosted and cut in small pieces after approx. 45 minutes, then ground by meat dicer affording 0.51 kg of ovary paste. The paste was then transferred into a 1 L reactor and 5 volumes (V) of solvent (MeOH+AcOH 0.5% v/v) was added for extraction. The mixture was stirred for 60 minutes at 200 rpm, after that it was discharged. After 45 minutes, the separation of the phases was observed. Despite this separation, the solid was again suspended by agitation of the mixture and the mixture was filtered on a 240 mm Buchner equipped with a por. 42 paper filter. A clear solution was collected after the filtration. The solid residue (approx. 350 g) was subjected to the second extraction step. For the second extraction, 5 volumes (V) of solvent (MeOH+AcOH 0.5% v/v) was added and the mixture was stirred for 60 minutes at 200 rpm. After the mixing, the separation of the phases was observed within 45 minutes, then the mixture was filtered on a 240 mm Buchner equipped with a por. 42 paper filter. A clear solution was collected after the filtration. No significant issues were observed during the filtration step, and 307 g of wet residue were isolated. After the two extractions, the recovery of TTX is R %=70% (TTX 1=33.5 ppm).

### Example 5-Extraction with MeOH/H₂O/AcOH (79:20:1% v/v)

0.51 kg (TTX=51 ppm) of pufferfish ovaries were defrosted and cut in small pieces after approx. 45 minutes, then ground by meat dicer affording 0.51 kg of ovary paste. The paste was then transferred in a 1 L reactor and 5 volumes (V) of solvent (MeOH/H₂O/AcOH, 79:20:1% v/v) were added for extraction. The mixture was stirred for 60 minutes at 200 rpm, after that it was discharged. After 45 minutes, the separation of the phases was observed. Despite this separation, the solid was again suspended by agitation and the mixture was filtered on a 240 mm Buchner equipped with a por. 42 paper filter. A clear solution was collected after the filtration. The solid residue (approx. 465 g) was subjected to a second extraction step. In this case, the filtration appeared slower than the other performed Trials, and it was completed in approx. 60 minutes. For the second extraction, 5 volumes (V) of solvent (MeOH/H₂O/AcOH, 79:20:1% v/v) were added and the mixture was stirred for 60 minutes at 200 rpm. After the mixing, the separation of the phases was observed within 45 minutes, then the mixture was filtered on a 240 mm Buchner equipped with a por. 42 paper filter. In this step, the filtration was extremely slow, and a break for the night was added: the mixture was recovered from the filter and stored overnight at 2-8° C. After the night, the mixture filtration was performed again, but even in this case, a slow process was observed. Taking into account the night stop, approx. 7 hours and 30 minutes were necessary for the filtration of the mixture. The resulting clear solution was collected and stored in fridge at 2-8° C.; moreover, 606 g of wet residue were isolated after the filtration. After the first extraction, the recovery of TTX is R %=70% (TTX=33.5 ppm). After the two extractions, the recovery of TTX is R %=94% (TTX=47.8 ppm).

### Example 6-Extraction with MeOH/H₂O/AcOH (79.5:20:0.5% v/v)

0.51 kg (TTX=39 ppm) of pufferfish ovaries were defrosted and cut in small pieces after approx. 45 minutes, then ground by meat dicer affording 0.51 kg of ovary paste. The paste was then transferred in a 1 L reactor and 5 volumes (V) of solvent (MeOH/H₂O/AcOH, 79.5:20:0.5% v/v) was added for extraction. The mixture was stirred for 60 minutes at 200 rpm, after that it was discharged. After 30 minutes, the separation of the phases was observed. Despite this separation, the solid was again suspended by agitation and the mixture was filtered on a 240 mm Buchner equipped with a por. 42 paper filter. A clear solution was collected after the filtration. The solid residue (approx. 405 g) was subjected to the second extraction step. For the second extraction, 5 volumes (V) of solvent (MeOH/H₂O/AcOH, 79.5:20:0.5% v/v) was added and the mixture was stirred for 60 minutes at 200 rpm. After the mixing, the separation of the phases was observed within 45 minutes, then the mixture was filtered on a 240 mm Buchner equipped with a por. 42 paper filter. Even in this case, during the second filtration some issues were faced. When the step was carried out, a slow filtration was initially observed. Again, a break for the night was introduced, so the mixture was recovered from the filter and stored overnight at 2-8° C. After the night, the filtration was again performed, but in this case no issues were observed and the filtration proceeded very efficiently. Taking into account the night break, the filtration was completed in approx. 90 minutes. The resulting clear solution was collected and stored in fridge at 2-8° C.; moreover, 404 g of wet residue were isolated after the filtration. After the two extractions, the recovery of TTX is quantitative, R %>95%.

### Example 7-Extraction with: step a) MeOH+0.5% v/v AcOH, step a1) MeOH/H₂O/AcOH (90:10:0.5% v/v)

3.78 kg (TTX=approx. 50 ppm) of pufferfish ovaries were defrosted by storing it at 5-10° C. overnight prior to be used. The mass was ground by meat dicer affording a compact ovary paste (3.70 kg). With the aim to enhance the homogeneity of the solid for assay determination sampling, the mass was further defrosted for addition approx. 45 minutes and manually mixed with a mechanical stirrer.

Step a)

A glass jacketed reactor was charged with (sorted by addition order):
Ovary paste, 3.7 kg;
Methanol, 18.5 L corresponding to 5 volumes (V);
Glacial acetic acid, 93 mL corresponding to 0.5% v/v.

The reactor was maintained at room temperature (20-25° C.) and the mass was stirred using a simple paddle at the reactor bottom. A stirring rate of 150 rpm was applied inducing a vorticose agitation. The ovary appeared to rapidly disperse as soon as the stirring was moved. In FIG. 6a-c, the suspension appearance is displayed. The suspension was maintained under stirring for 60 minutes, then the suspension was left to spontaneously decant, monitoring the phase separation overtime to qualitatively assess the relative volume of the settled solid. After overnight sedimentation, a volumetric ratio of solution: solid of approx. 3.2 was observed, then the liquid phase was filtered on a 240 mm Buchner equipped with a por. 42 paper filter. The withdrawing was performed using a peristaltic pump, while the solid residue (approx. 7 L) was left in the reactor for step a1). A clear solution (12.1 kg, measured density=0.839 g/mL corresponding to 14.4 L, pH approx. 4.3-4.6) was collected after the filtration and was transferred into a second jacketed reactor equipped with a distillation apparatus to concentrate the solution. The concentration step was performed by heating the solution at 50° C. (T jacket) and applying a vacuum of approx. 200 mbar; the stirring was set at 100 rpm during the distillation. After approx. 26 hours, the resulting concentrated solution was discharged from the reactor (1.877 kg, measured density=0.925 g/mL corresponding to 2.03 L, pH approx. 4.3-4.6). The solution was concentrated by a factor 6.4 w/w (7.1 v/v).

Step a1

To the solid residue (20 L volume) resulting from step a) (approx. 7 L), extraction solvents were added as follows (total volume 14.0 L):
Methanol, 12.6 L, corresponding to 90% v/v;
Water, 1.4 L corresponding to 10% v/v;
Glacial acetic acid, 69 mL corresponding to 0.5% v/v.

The temperature of the reactor was monitored (20-25° C.) and the mass was stirred using a simple paddle at the reactor bottom. A stirring rate of 150 rpm was applied inducing a vorticose agitation. The suspension was maintained under stirring for 60 minutes, then the suspension was left to spontaneously decant overnight. After overnight sedimentation, a volumetric ratio solution/solid of approx. 3.2 was observed, then the liquid phase was withdrawn using a peristaltic pump and filtered on a 240 mm Buchner equipped with a por. 42 paper filter. A clear solution (13.92 kg, measured density=0.833 g/mL corresponding to 16.7 L, pH approx. 4.3-4.6) was collected after the filtration and was transferred to reactor for the concentration step. 2.5 kg of residual biomass were otherwise discharged from the reactor (filtered with a qualitative paper filter) and stored in fridge. The concentration step was performed by heating the solution at 50° C. (T jacket) and applying a vacuum of approx. 200 mbar; the stirring was set at 100 rpm during the distillation. After approx. 19.5 hours, the resulting concentrated solution was discharged from the reactor (1.94 kg, measured density=0.923 g/mL corresponding to 2.1 L, pH approx. 4.3-4.6). The solution was concentrated by a factor 7.2 w/w (7.9 v/v). After the recovery of the concentrated solution, the formation of a gelatinous solid was observed at the bottom of the solution. The resulting solid was isolated by filtration (Whatman™ paper filter porosity 42-2.5 μm) (60 g). After steps a) and a1), the recovery of TTX was R %=70% (TTX 1=34.4 ppm).

### Example 8—Extraction with step a) MeOH+0.5% v/v AcOH, step a1) MeOH/H₂O/AcOH (90:10:0.5% v/v)

2.15 kg (TTX=42 ppm) of pufferfish ovaries were defrosted, cut, loaded into a reactor and extracted with 10.75 L (5V) of MeOH +AcOH 0.5% v/v; the mixture was kept stirred at RT for approx. 1 h and left in decantation for at least 4 h; the solution was separated from the solid by sucking under vacuum, while the latter was kept in the reactor and extracted again with 8 L (3.75 vol.) of a mixture of MeOH/H₂O 90/10 v/v +AcOH 0.5% v/v, following the same procedure, but keeping in decantation without stirring overnight. The liquid phase was separated, pooled with that coming from the first extraction and filtered on a Buchner equipped with a por. 42 paper filter. The filtered solution, V=17.5 L, was concentrated till V=1.7 L.

### Example 9—Extraction with n-Hexane of the Extracting Solution Prepared According to Example 4

A fraction of the extracting solution obtained after the extraction, prepared according to Example 4 (approx. 800 mL) was placed in a 1 L reactor and then concentrated under vacuum (approx. 100 mbar) at 50° C. (T jacket) until a final volume of approx. 100 mL was reached. At this time, the concentrate was washed with 1 volume (100 mL) of hexane in a separation funnel, then the aqueous phase was recovered (76.2 g) and stored in fridge. After phase separation, the dry residue in the organic phase is approx. DR %=3.6% of the dry residue of the extraction solution.

### Example 10—Extraction with n-Hexane of the Extracting Solution Prepared according to Example 6

A fraction of the extracting solution obtained after the extraction prepared according to Example 6 (approx. 800 mL) was placed in a 1L reactor and then concentrated under vacuum (approx. 100 mbar) at 50° C. (T jacket) until a final volume of approx. 100 mL was reached. At this time, the concentrate was washed with 1 volume (100 mL) of hexane in a separation funnel, then the aqueous phase was recovered (93 g) and stored in fridge. After phases separation, the dry residue in the organic phase is approx. DR %=3.1% of the dry residue of the extraction.

### Example 11—Extraction with Dichloromethane: Methanol and n-Butanol of the Extracting Solution According to Example 8

The filtered, concentrated solution (1.7 L) of Example 8 was extracted twice with a mixture of dichloromethane/methanol 90/10 v/v (0.9 L×2) and the organic phases were eliminated. The aqueous phase, V=1.55 L, was concentrated till V=0.85 L (max vacuum, T ext.=50° C.); then 0.5 L of n-BuOH were added and the mixture was concentrated till V=300 mL. The concentrated solution was extracted 4 times with n-BuOH (1st=300 mL, 2nd=250 mL; 3rd=250 ml; 4th=180 mL) and the aqueous phase was concentrated to dryness (weight=60 g). The resulting material (60 g) is pre-purified TTX.

### Example 12—Purification on Si Gel 60 of Pre-Purified TTX

The pre-purified TTX (60 g) resulting from Example 12 was suspended with MeOH (480 mL=8V) and loaded onto a Si gel 60 glass column (900 g=15 parts vs load weight; [symbol] =8 cm, height Si-gel=48 cm) previously packed with a MeOH/EtOAc 1/1 mixture. TTX was eluted (elution flow=1.5 L/h, linear speed=30 cm/h) adopting the following gradient mixture: MeOH/EtOAc 1/1 (5 L), EtOAc/MeOH/H₂O 4/4/2 (4.5 L), EtOAc/MeOH/H₂O 4/4/2+5% AcOH (8 L) MeOH/H₂O 8/2+5% AcOH (8 L). The fractions containing TTX were collected and concentrated to dryness to give a pasty brown oil=50 g.

### Example 13—Preparative HPLC RP18 Column

The pasty oil coming from Example 13 was dissolved with H₂O+1% AcOH till V=80 mL and further diluted with the same mixture until V=200 mL (4V vs oil weight). The diluted solution was loaded, in aliquots of 1 mL, on a RP-HPLC column (Agilent Zorbax™ SB300-C18 250×21.2, 7 μm) and purified according to the following purification HPLC parameters provided in Table 9.

TABLE 8
Mobile phase gradient:

| | | | 8 mM Sodium 1- | | |
|---|---|---|---|---|---|
| | | | Heptansulfonate + | | |
| | Time | Flow (mL/min) | 50 ppm TFA | acetonitrile | water |
| 1 | 0 | 21.0 | 100.0 | 0.0 | 0.0 |
| 2 | 23 | 21.0 | 100.0 | 0.0 | 0.0 |
| 3 | 25 | 21.0 | 0.0 | 0.0 | 100.0 |
| 4 | 26 | 21.0 | 0.0 | 10.0 | 90.0 |
| 5 | 32 | 21.0 | 0.0 | 90.0 | 10.0 |
| 6 | 34 | 21.0 | 0.0 | 90.0 | 10.0 |
| 7 | 36 | 21.0 | 0.0 | 10.0 | 90.0 |
| 8 | 38 | 21.0 | 100.0 | 0.0 | 0.0 |
| 9 | 45 | 21.0 | 100.0 | 0.0 | 0.0 |

The heart fractions (collected in the window between 12-17.5 minutes) from 200 preparative runs were mixed together and concentrated through a freeze dryer firstly till V=1.2 L and, finally, till V=100 mL.

### Example 14—Preparative CN-HPLC-for Removal of the Ion Pairing Agent

An aliquot corresponding to (15 mL) of the fraction obtained from example 14 was loaded, in aliquots of 0.6 mL, on a CN-HPLC column (ADAMAS CN, 250×21.2 mm×ID 5 μm) and purified to remove ion pairing agent according to the following preparative HPLC purification parameters: Eluent: H₂O+600 ppm NH₃33%+50 ppm HCOOH. The heart fractions (collected between 8.5 and 15.5 min) from 25 preparative runs were mixed together and concentrated through a freeze dryer firstly till V=200 mL and finally till V=20 L.

Although the above description includes reference to certain specific embodiments, various modifications thereof will be apparent to those skilled in the art. Any examples provided herein are included solely for the purpose of illustration and are not intended to be limiting in any way. Any drawings provided herein are solely for the purpose of illustrating various aspects of the description and are not intended to be drawn to scale or to be limiting in any way. The scope of the claims appended hereto should not be limited by the preferred embodiments set forth in the above description but should be given the broadest interpretation consistent with the present specification as a whole. The disclosures of all prior art recited herein are incorporated herein by reference in their entirety.

1. A process for extracting tetrodotoxin (TTX) from biological material containing TTX, the process comprising:
a) extracting the TTX from the biological material with a first organic solvent (OS 1) to provide a first extraction solid residue (ESR 1) and a first liquid phase (LP 1);
b) concentrating the LP 1 to provide a concentrated liquid phase (C-LP 1);
c) washing of C-LP 1 with a second organic solvent (OS 2) to provide a second liquid phase (LP 2) and a washing phase (WP 1);
d) concentrating the LP 2 to provide a pre-purified TTX (PP-TTX); and, e) purifying the PP-TTX by column chromatography on silica gel to provide crude TTX (C-TTX).

2. The process of claim 1, wherein the first or second organic solvent is one or more of acetone, acetonitrile, benzene, n-butanol, n-butyl acetate, carbon tetrachloride, chloroform, cyclohexane, 1,2-dichloroethane dichloromethane, diethyl ether, isopropyl ether, dimethylformamide, dimethyl sulfoxide, dioxane, ethanol, ethyl acetate, heptane, hexane, isooctane, isopropanol, methanol, methyl ethyl ketone, methyl tert-butyl ether, nitromethane pentane, 1-propanol, tetrahydrofuran, toluene, trichloroethylene, and xylene, or a mixture thereof with water.

3. The process of claim 1, wherein the first and/or second organic solvent further comprises an organic acid.

4. The process of claim 3, wherein the organic acid is present at a concentration of less than about 5% of the total volume of the first or second organic solvent, respectively.

5. The process claim 1, wherein the organic acid is one or more of formic acid, acetic acid, trifluoroacetic acid, propionic acid, pentanoic acid, hexanoic acid, butyric acid, sorbic acid, lactic acid, citric acid, ascorbic acid, fumaric acid, malic acid, tartaric acid, oxalic acid, citric acid, gluconic acid, glutaric acid, glutamic acid, benzoic acid, benzylic acid, acetylsalicylic acid, cinnamic acid, gallic acid and acetic acid.

6. The process of claim 1, wherein the first organic solvent and second organic solvent each have a water content less than 40%.

7. The process of claim 1 wherein the first organic solvent is different than the second organic solvent.

8. The process of claim 1, wherein step (a) is repeated at least once.

9. A process for extracting TTX from a biological material containing TTX, the process comprising:
a) extracting the TTX from the biological material with a first organic solvent (OS 1) to provide a first extraction solid residue (ESR 1) and a first liquid phase (LP 1);
a1) extracting the ESR 1 with a further organic solvent (OS 3) to provide a further extraction solid residue (ESR 2) and a further liquid phase (LP 3);
a2) combining LP1 and LP 3 to provide a combined liquid phase (LP 4);
b) concentrating the LP 4 to provide a concentrated liquid phase (C-LP 4);
c) washing the C-LP 4 with a second organic solvent (OS 2) to provide a second liquid phase (LP 2) and a washing phase (WP 1);
d) concentrating the LP 2 to provide a pre-purified TTX (PP-TTX); and
e) purifying the PP-TTX by column chromatography on silica gel to provide crude TTX (C-TTX).

10. The process of claim 9 further comprising:
f) purifying the C-TTX by preparative high performance liquid chromatography to provide purified TTX (P-TTX).

11. The process of claim 9, wherein at least one of steps (a), (a1), and (c) is repeated at least once.

12. The process of claim 9, wherein OS 2 is one or more of: benzene, n-butanol, n-butyl acetate, carbon tetrachloride, chloroform, cyclohexane, 1,2-dichloroethane dichloromethane, diethyl ether, diisopropyl ether, ethyl acetate, heptane, hexane, isooctane, methanol, methyl ethyl ketone, methyl tert-butyl ether, nitromethane pentane, toluene, trichloroethylene, and xylene.

13. The process of claim 9, wherein OS 2 is one or more of methanol, dichloromethane, hexane, and butanol.

14. The process of claim 9, wherein step c) is repeated at least once.

15. The process of any one of claim 10, wherein step f) comprises the use of a solvent, and wherein the solvent includes an ion pairing agent, wherein the ion pairing agent is one or more of sodium 1-propanesulfonate, sodium 1-butanesulfonate, sodium 1-pentansulfonate, sodium 1-hexanesulfonate, sodium 1-heptanesulfonate, sodium 1-octanesulfonate, sodium 1-nonanesulfonate, sodium 1-decanesulfonate, sodium 1-undecanesulfonate, sodium 1-dodecanesulfonate, sodium 1-tridecanesulfonate, sodium dodecyl sulfate, tetraethylammonium hydroxide, tetrapropylammonium hydroxide, tetrabutylammonium hydroxide, tetrabutylammonium bromide, tetrabutylammonium chloride, tetrabutylammonium phosphate, tetrabutylammonium hydrogen sulfate, dodecyltrimethylammonium chloride, and tetra (decyl) ammonium bromide.

16. The process of 15 further comprising:
g) purifying the P-TTX 1 by high performance liquid chromatography to remove the ion pairing agent.

17. The process of claim 16, wherein the purifying of P-TTX comprises use of one or more of methanol, ethanol, propanol, iso-propanol, acetone, acetonitrile, water, and ethyl acetate as the mobile phase.

18. The process of claim 17, wherein the mobile phase further contains:
an organic acid, wherein the organic acid is one or more of formic acid, acetic acid and trifluoroacetic acid; and
an organic base, wherein the organic base is one or more of triethylamine, diethylamine, and ammonia.

19. The process of claim 9 wherein the first solvent OS 1 and the third solvent OS 3 each independently comprise one or more of acetone, acetonitrile, benzene, n-butanol, n-butyl acetate, carbon tetrachloride, chloroform, cyclohexane, 1,2-dichloroethane dichloromethane, diethyl ether, isopropyl ether, dimethylformamide, dimethyl sulfoxide, dioxane, ethanol, ethyl acetate, heptane, hexane, isooctane, isopropanol, methanol, methyl ethyl ketone, methyl tert-butyl ether, nitromethane pentane, 1-propanol, tetrahydrofuran, toluene, trichloroethylene, and xylene, or a mixture thereof with water.

20. The process of claim 19 wherein each of OS 1, OS 2, and OS 3 are different from each other.

21. (canceled)
